(19) United States
(12) Patent Application Publication
(10) Pub. No.: US 2005/0255050 A1
(43) Pub. Date: Nov. 17, 2005

(54) Powder formulations for inhalation, comprising enantiomerically pure beta agonists

(75) Inventors: Michael Josef Friedrich Trunk, Ingelheim (DE); Joerg Schiewe, Mainz (DE)

Correspondence Address: MICHAEL P. MORRIS;BOEHRINGER INGELHEIM CORPORATION, 900 RIDGEBURY ROAD, P. O. BOX 368, RIDGEFIELD, CT, 06877-0368, US

(73) Assignee: Boehringer Ingelheim International GmbH, Ingelheim (DE)

(21) Appl. No.: 11/128,141

(22) Filed: May 12, 2005

Related U.S. Application Data
(60) Provisional application No. 60/578,527, filed on Jun. 10, 2004.

(30) Foreign Application Priority Data
May 14, 2004 (DE) 10 2004 024 451

(51) Int. Cl. A61L 9/04; A61K 31/538

(52) U.S. Cl. 424/46; 514/230.5

(57) ABSTRACT

The present invention relates to powder formulations for inhalation containing enantiomerically pure compounds of general formula 1 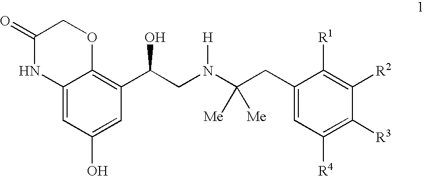
wherein the groups R¹, R², R³ and R⁴ may have the meanings given in the claims and in the specification, optionally in the form of the pharmaceutically acceptable acid addition salts thereof, as well as optionally in combination with a pharmaceutically acceptable excipient, processes for preparing them and their use as pharmaceutical compositions, particularly as pharmaceutical compositions for the treatment of respiratory complaints.

This application claims priority benefit under 35 USC 119 (e) to U.S. Provisional Application 60/578,527, filed Jun. 10, 2004.

The present invention relates to powder formulations for inhalation, containing enantiomerically pure compounds of general formula 1 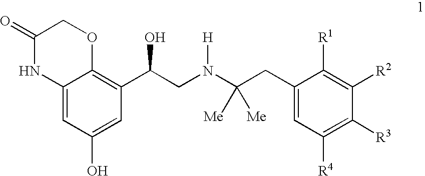
wherein the groups R₁, R², R³ and R⁴ may have the meanings given in the claims and in the specification, optionally in the form of the pharmaceutically acceptable acid addition salts thereof, as well as optionally in combination with a pharmaceutically acceptable excipient, processes for preparing them and their use as pharmaceutical compositions, particularly as pharmaceutical compositions for the treatment of respiratory complaints

### BACKGROUND TO THE INVENTION

Betamimetics (β-adrenergic substances) are known from the prior art. For example reference may be made in this respect to the disclosure of U.S. Pat. No. 4,460,581, which proposes betamimetics for the treatment of a range of diseases.

For drug treatment of diseases it is often desirable to prepare medicaments with a longer duration of activity. As a rule, this ensures that the concentration of the active substance in the body needed to achieve the therapeutic effect is guaranteed for a longer period without the need to re-administer the drug at frequent intervals. Moreover, giving an active substance at longer time intervals contributes to the well-being of the patient to a high degree.

In a particularly preferred embodiment the present invention relates to pharmaceutical preparations which may confer a therapeutic benefit in the treatment of respiratory complaints.

Particularly for treating respiratory complaints, it is useful to administer the active substance by inhalation. In addition to the administration of broncholytically active compounds in the form of metered aerosols and inhalable solutions, the use of inhalable powders containing active substance is of particular importance.

With active substances which have a particularly high efficacy, only small amounts of the active substance are needed per single dose to achieve the desired therapeutic effect. In such cases, the active substance has to be diluted with suitable excipients in order to prepare the inhalable powder. Because of the large amount of excipient, the properties of the inhalable powder are critically influenced by the choice of excipient. When choosing the excipient its particle size is particularly important. As a rule, the finer the excipient, the poorer its flow properties. However, good flow properties are a prerequisite for highly accurate metering when packing and dividing up the individual doses of preparation, e.g. when producing capsules (inhalettes) for powder inhalation or when the patient is metering the individual dose before using a multi-dose inhaler. Moreover, the particle size of the excipient is very important for the emptying characteristics of capsules when used in an inhaler. It has also been found that the particle size of the excipient has a considerable influence on the proportion of active substance in the inhalable powder which is delivered for inhalation. The term inhalable proportion of active substance refers to the particles of the inhalable powder which are conveyed deep into the branches of the lungs when inhaled with a breath. The particle size required for this is between 0.5 and 10 μm, preferably between 1 and 6 μm.

The aim of the invention is to prepare an inhalable powder containing a betamimetic which, while being accurately metered (in terms of the amount of active substance and powder mixture packed into each batch of powder by the manufacturer or by the patient before use, depending on the inhaler, as well as the quantity of active substance released and delivered to the lungs on each actuation by the inhalation process) with only slight variations between batches, enables the active substance to be administered in a large inhalable proportion. A further aim of the present invention is to prepare an inhalable powder containing a betamimetic which ensures good emptying characteristics of the capsules, whether it is administered to the patient using an inhaler, for example, as described in WO 94/28958, or in vitro using an impactor or impinger.

The fact that betamimetics have a high therapeutic efficacy even at very low doses imposes further conditions on an inhalable powder which is to be used with highly accurate metering. Because only a low concentration of the active substance is needed in the inhalable powder to achieve the therapeutic effect, a high degree of homogeneity of the powder mixture and only slight fluctuations in the dispersion characteristics from one batch of powder to the next are essential. The homogeneity of the powder mixture and minor fluctuations in the dispersion properties are crucial in ensuring that the inhalable proportion of active substance is released reproducibly in constant amounts and with the lowest possible variability.

Accordingly, a further aim of the present invention is to prepare an inhalable powder containing a betamimetic which is characterised by a high degree of homogeneity and uniformity of dispersion. The present invention also sets out to provide an inhalable powder which allows the inhalable proportion of active substance to be administered with the lowest possible variability.

The characteristics of emptying from the powder reservoir (the container from which the inhalable powder containing the active substance is released for inhalation) play an important part, not exclusively, but especially in the administration of inhalable powders using capsules containing powder. If only a small amount of the powder formulation is released from the powder reservoir as a result of minimal or poor emptying characteristics, significant amounts of the inhalable powder containing the active substance are left in the powder reservoir (e.g. the capsule or other container) and are unavailable to the patient for therapeutic use. The result of this is that the dosage of active substance in the powder mixture has to be increased so that the quantity of active substance delivered is sufficient to produce the desired therapeutic effect.

Against this background the present invention further sets out to provide an inhalable powder which is also characterised by very good emptying characteristics.

### DESCRIPTION OF THE INVENTION

The present invention relates to inhalable powders containing one or more, preferably one enantiomerically pure compound of general formula 1 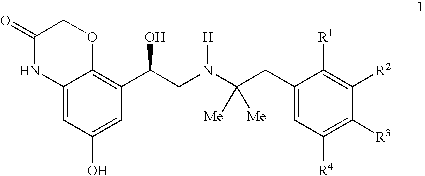
wherein
R¹ denotes hydrogen, C₁-C₄-alkyl, C₁-C₄-alkoxy or halogen;
R² denotes hydrogen, C₁-C₄-alkyl, C₁-C₄-alkoxy or halogen;
R³ denotes hydrogen, C₁-C₄-alkyl, C₁-C₄-alkoxy, halogen, OH, —O—C₁-C₄-alkylene-COOH or —O—C₁-C₄-alkylene-COO—C₁-C₄-alkyl;
R⁴ denotes hydrogen, C₁-C₄-alkyl, C₁-C₄-alkoxy or halogen,
   optionally in the form of the pharmaceutically acceptable acid addition salts, hydrates or solvates thereof, optionally in admixture with one or more physiologically acceptable excipients.

Preferred inhalable powders as mentioned above are those which contain one or more, preferably one enantiomerically pure compound of general formula 1, wherein
R¹ denotes hydrogen or halogen;
R² denotes hydrogen or halogen;
R³ denotes hydrogen, C₁-C₄-alkoxy or halogen;
R⁴ denotes hydrogen or halogen;
   optionally in the form of the pharmaceutically acceptable acid addition salts, hydrates or solvates thereof, optionally in admixture with one or more physiologically acceptable excipients.

Preferred inhalable powders are those which contain one or more, preferably one enantiomerically pure compound of general formula 1 contain, wherein
R¹ denotes hydrogen, fluorine or chlorine, preferably hydrogen or fluorine;
R² denotes hydrogen, fluorine or chlorine, preferably hydrogen or fluorine;
R³ denotes hydrogen, methoxy, ethoxy, fluorine or chlorine, preferably hydrogen, methoxy, ethoxy or fluorine;
R⁴ denotes hydrogen, fluorine or chlorine, preferably hydrogen or fluorine;
   optionally in the form of the pharmaceutically acceptable acid addition salts, hydrates or solvates thereof, optionally in admixture with one or more physiologically acceptable excipients.

Preferred inhalable powders are those which contain one or more, preferably one enantiomerically pure compound of general formula 1 contain, wherein
R¹ denotes hydrogen or fluorine;
R² denotes hydrogen;
R³ denotes methoxy, ethoxy or fluorine;
R⁴ denotes hydrogen;
   optionally in the form of the pharmaceutically acceptable acid addition salts, hydrates or solvates thereof, optionally in admixture with one or more physiologically acceptable excipients.

Of equal importance according to the invention are also inhalable powders which contain one or more, preferably one enantiomerically pure compound of general formula 1, wherein
R¹ denotes hydrogen;
R² denotes hydrogen, fluorine or chlorine, preferably hydrogen or fluorine;
R³ denotes hydrogen;
R⁴ denotes hydrogen, fluorine or chlorine, preferably hydrogen or fluorine;
   optionally in the form of the pharmaceutically acceptable acid addition salts, hydrates or solvates thereof, optionally in admixture with one or more physiologically acceptable excipients.

Also preferred according to the invention are inhalable powders which contain one or more, preferably one enantiomerically pure compound of general formula 1 in the form of the free bases thereof.

Of equal importance according to the invention are also inhalable powders which contain one or more, preferably one enantiomerically pure compound of general formula 1 in the form of the pharmaceutically acceptable acid addition salts thereof, which may be represented by general formula 1-HX.

Preferred inhalable powders contain as acid addition salts one or more, preferably one compound of general formula 1-HX, 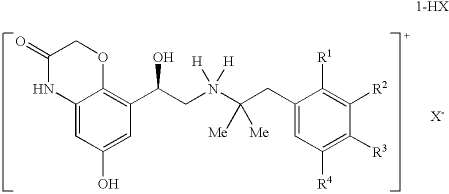
wherein
X⁻ denotes an anion with a single negative charge, preferably an anion with a single negative charge selected from the group consisting of chloride, bromide, iodide, sulphate, phosphate, methanesulphonate, nitrate, maleate, acetate, benzoate, citrate, salicylate, trifluoroacetate, fumarate, tartrate, oxalate, succinate, benzoate and p-toluenesulphonate,
   and the groups R₁, R², R³ and R⁴ may have one of the above-mentioned meanings, optionally in the form of the tautomers, mixtures of the tautomers, hydrates or solvates thereof, as well as optionally in admixture with one or more physiologically acceptable excipients.

Preferred inhalable powders contain one or more, preferably one compound of formula 1-HX, wherein
X⁻ denotes an anion with a single negative charge selected from the group consisting of chloride, bromide, sulphate, methanesulphonate, maleate, acetate, benzoate, citrate, salicylate, trifluoroacetate, fumarate, tartrate and succinate;
   and the groups R¹, R², R³ and R⁴ may have one of the above-mentioned meanings, optionally in the form of the tautomers, mixtures of the tautomers, hydrates or solvates thereof, as well as optionally in admixture with one or more physiologically acceptable excipients.

Preferred inhalable powders contain one or more, preferably one compound of formula 1-HX, wherein
X⁻ denotes an anion with a single negative charge selected from the group consisting of chloride, methanesulphonate, maleate, acetate, citrate, salicylate, trifluoroacetate, fumarate and succinate, preferably chloride, maleate, salicylate, fumarate and succinate, particularly preferably chloride;
   and the groups R¹, R², R³ and R⁴ may have one of the above-mentioned meanings, optionally in the form of the tautomers, mixtures of the tautomers, hydrates or solvates thereof, as well as optionally in admixture with one or more physiologically acceptable excipients.

Also particularly preferred are inhalable powders which contain one or more, preferably one enantiomerically pure compound of general formula 1 which are selected from the group consisting of
6-hydroxy-8-{(R)-1-hydroxy-2-[2-(4-methoxy-phenyl)-1,1-dimethyl-ethylamino]-ethyl}-4H-benzo[1,4]oxazin-3-one-hydrochloride;
8-{(R)-2-[2-(2,4-difluoro-phenyl)-1,1-dimethyl-ethylamino]-1-hydroxy-ethyl}-6-hydroxy-4H-benzo[1,4]oxazin-3-one-hydrochloride;
8-{(R)-2-[2-(3,5-difluoro-phenyl)-1,1-dimethyl-ethylamino]-1-hydroxy-ethyl}-6-hydroxy-4H-benzo[1,4]oxazin-3-one-hydrochloride;
8-{(R)-2-[2-(4-ethoxy-phenyl)-1,1-dimethyl-ethylamino]-1-hydroxy-ethyl}-6-hydroxy-4H-benzo[1,4]oxazin-3-one-hydrochloride;
8-{(R)-2-[2-(4-fluoro-phenyl)-1,1-dimethyl-ethylamino]-1-hydroxy-ethyl}-6-hydroxy-4H-benzo[1,4]oxazin-3-one-hydrochloride;
6-hydroxy-8-{(R)-1-hydroxy-2-[2-(4-methoxy-phenyl)-1,1-dimethyl-ethylamino]-ethyl}-4H-benzo[1,4,]oxazin-3-one maleate;
6-hydroxy-8-{(R)-1-hydroxy-2-[2-(4-methoxy-phenyl)-1,1-dimethyl-ethylamino]-ethyl}-4H-benzo[1,4,]oxazin-3-one salicylate;
6-hydroxy-8-{(R)-1-hydroxy-2-[2-(4-methoxy-phenyl)-1,1-dimethyl-ethylamino]-ethyl}-4H-benzo[1,4,]oxazin-3-one succinate;
6-hydroxy-8-{(R)-1-hydroxy-2-[2-(4-methoxy-phenyl)-1,1-dimethyl-ethylamino]-ethyl}-4H-benzo[1,4,]oxazin-3-one-fumarate;
8-{(R)-2-[2-(2,4-difluoro-phenyl)-1,1-dimethyl-ethylamino]-1-hydroxy-ethyl}-6-hydroxy-4H-benzo[1,4]oxazin-3-one-maleate;
8-{(R)-2-[2-(2,4-difluoro-phenyl)-1,1-dimethyl-ethylamino]-1-hydroxy-ethyl}-6-hydroxy-4H-benzo[1,4]oxazin-3-one-salicylate;
8-{(R)-2-[2-(2,4-difluoro-phenyl)-1,1-dimethyl-ethylamino]-1-hydroxy-ethyl}-6-hydroxy-4H-benzo[1,4]oxazin-3-one-succinate;
8-{(R)-2-[2-(2,4-difluoro-phenyl)-1,1-dimethyl-ethylamino]-1-hydroxy-ethyl}-6-hydroxy-4H-benzo[1,4]oxazin-3-one-fumarate;
8-{(R)-2-[2-(3,5-difluoro-phenyl)-1,1-dimethyl-ethylamino]-1-hydroxy-ethyl}-6-hydroxy-4H-benzo[1,4]oxazin-3-one-maleate;
8-{(R)-2-[2-(3,5-difluoro-phenyl)-1,1-dimethyl-ethylamino]-1-hydroxy-ethyl}-6-hydroxy-4H-benzo[1,4]oxazin-3-one-salicylate;
8-{(R)-2-[2-(3,5-difluoro-phenyl)-1,1-dimethyl-ethylamino]-1-hydroxy-ethyl}-6-hydroxy-4H-benzo[1,4]oxazin-3-one-succinate;
8-{(R)-2-[2-(3,5-difluoro-phenyl)-1,1-dimethyl-ethylamino]-1-hydroxy-ethyl}-6-hydroxy-4H-benzo[1,4]oxazin-3-one-fumarate;
8-{(R)-2-[2-(4-ethoxy-phenyl)-1,1-dimethyl-ethylamino]-1-hydroxy-ethyl}-6-hydroxy-4H-benzo[1,4]oxazin-3-one-maleate;
8-{(R)-2-[2-(4-ethoxy-phenyl)-1,1-dimethyl-ethylamino]-1-hydroxy-ethyl}-6-hydroxy-4H-benzo[1,4]oxazin-3-one-salicylate;
8-{(R)-2-[2-(4-ethoxy-phenyl)-1,1-dimethyl-ethylamino]-1-hydroxy-ethyl}-6-hydroxy-4H-benzo[1,4]oxazin-3-one-succinate;
8-{(R)-2-[2-(4-ethoxy-phenyl)-1,1-dimethyl-ethylamino]-1-hydroxy-ethyl}-6-hydroxy-4H-benzo[1,4]oxazin-3-one-fumarate;
8-{(R)-2-[2-(4-fluoro-phenyl)-1,1-dimethyl-ethylamino]-1-hydroxy-ethyl}-6-hydroxy-4H-benzo[1,4]oxazin-3-one-maleate;
8-{(R)-2-[2-(4-fluoro-phenyl)-1,1-dimethyl-ethylamino]-1-hydroxy-ethyl}-6-hydroxy-4H-benzo[1,4]oxazin-3-one-salicylate;
8-{(R)-2-[2-(4-fluoro-phenyl)-1,1-dimethyl-ethylamino]-1-hydroxy-ethyl}-6-hydroxy-4H-benzo[1,4]oxazin-3-one-succinate and
8-{(R)-2-[2-(4-fluoro-phenyl)-1,1-dimethyl-ethylamino]-1-hydroxy-ethyl}-6-hydroxy-4H-benzo[1,4]oxazin-3-one-fumarate,
   optionally in the form of the tautomers, mixtures of the tautomers, hydrates or solvates thereof.

In the inhalable powders according to the invention the enantiomerically pure compounds of general formula 1, wherein R₁, R², R³ R⁴ are as hereinbefore defined are present in crystalline form, optionally in the form of the crystalline tautomers, crystalline hydrates or crystalline solvates thereof. Particularly preferred are enantiomerically pure, crystalline compounds of general formula 1 wherein R¹, R², R³ and R⁴ have the meanings given above, optionally in the form of the crystalline tautomers, crystalline hydrates or crystalline solvates thereof, which are further characterised in that they are crystalline compounds which occur in only one crystal modification.

By the expression “only one crystal modification” are meant crystalline compounds of formula 1 which do not constitute a mixture of any polymorphic crystal modifications which may exist and/or mixtures of one or more crystal modifications with the amorphous or vitreous state of the compounds according to formula 1.

Unless otherwise stated, the alkyl groups are straight-chained or branched alkyl groups having 1 to 4 carbon atoms. The following are mentioned by way of example: methyl, ethyl, propyl or butyl. In some cases the abbreviations Me, Et, Prop or Bu are used to denote the groups methyl, ethyl, propyl or butyl. Unless otherwise stated, the definitions propyl and butyl include all the possible isomeric forms of the groups in question. Thus, for example, propyl includes n-propyl and iso-propyl, butyl includes iso-butyl, sec.butyl and tert.-butyl, etc.

Unless otherwise stated, the alkylene groups are branched and unbranched double-bonded alkyl bridges having 1 to 4 carbon atoms. The following are mentioned by way of example: methylene, ethylene, n-propylene or n-butylene.

Unless otherwise stated, the term alkyloxy groups (or —O-alkyl groups) denotes branched and unbranched alkyl groups having 1 to 4 carbon atoms which are linked via an oxygen atom. Examples of these include: methyloxy, ethyloxy, propyloxy or butyloxy. The abbreviations MeO—, EtO—, PropO— or BuO— are used in some cases to denote the groups methyloxy, ethyloxy, propyloxy or butyloxy. Unless otherwise stated, the definitions propyloxy and butyloxy include all possible isomeric forms of the groups in question. Thus, for example, propyloxy includes n-propyloxy and iso-propyloxy, butyloxy includes iso-butyloxy, sec.butyloxy and tert.-butyloxy, etc. In some cases, within the scope of the present invention, the term alkoxy is used instead of the term alkyloxy. Accordingly, the terms methoxy, ethoxy, propoxy or butoxy may also be used to denote the groups methyloxy, ethyloxy, propyloxy or butyloxy.

Halogen within the scope of the present invention denotes fluorine, chlorine, bromine or iodine. Unless stated to the contrary, fluorine, chlorine and bromine are the preferred halogens.

The term enantiomerically pure describes within the scope of the present invention compounds of formula 1 which are present in an enantiomerical purity of at least 85% ee, preferably at least 90% ee, particularly preferably ≧95% ee. The term ee (enantiomeric excess) is known in the art and describes the optical purity of chiral compounds.

The highly crystalline compounds of formula 1 may be obtained as illustrated below (Diagram 1). 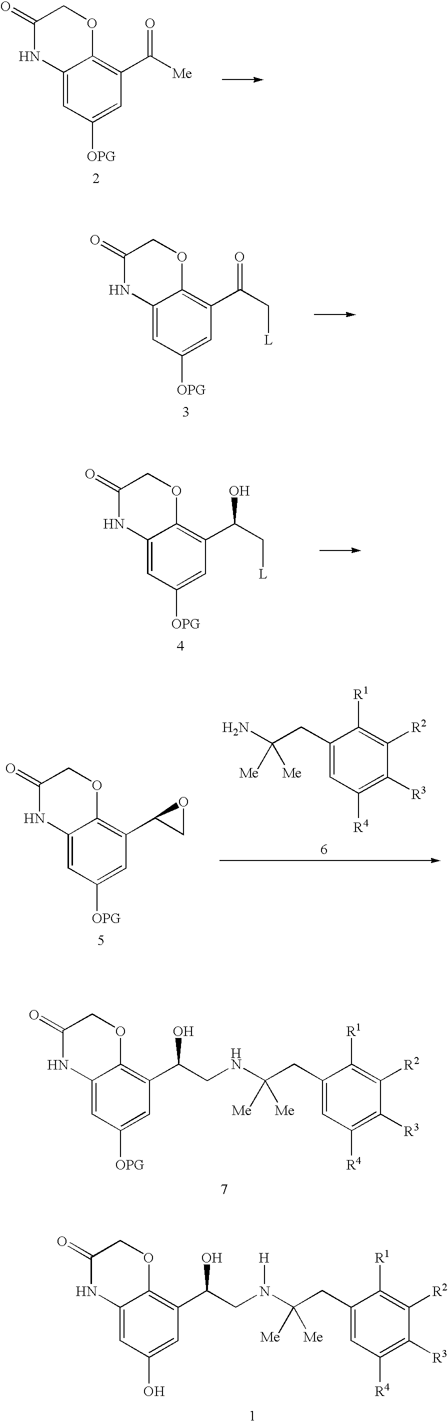
Diagram 1:

In the compounds of formulae 2 to 5 and 7 mentioned in Diagram 1 the group OPG denotes a hydroxyl function protected by a protective group (PG). For a choice of suitable protective groups for the hydroxyl group reference is made to the prior art as given for example in Protective Groups in Organic Synthesis, T. W. Greene and P. G. M. Wuts, John Wiley & Sons Inc, third Edition, 1999.

Preferably OPG denotes a group which is selected from among
—O—C₁-C₄-alkyl, —O-benzyl or —O—CO—C₁-C₄-alkyl, preferably
—O-methyl, -, —O-benzyl or —O-acetyl, particularly preferably —O-methyl or
—O-benzyl, particularly preferably —O-benzyl.

In the compounds of formulae 3 and 4 mentioned in Diagram 1 the group L denotes a leaving group. Preferably L denotes a leaving group selected from among
chlorine, bromine, iodine, methanesulphonate, trifluoromethanesulphonate and
p-toluenesulphonate, preferably chlorine or bromine, particularly preferably chlorine.

In the compounds of formulae 6 and 7 mentioned in Diagram 1 the groups R¹, R², R³ and R⁴ may have the above-mentioned meanings.

Starting from 8-acetyl-6-benzyloxy-4H-benzo[1,4]oxazin-3-one (2) the compounds of general formula 3 are prepared in the manner known from the prior art. The compound of formula 3 is then converted enantioselectively, in the presence of a chiral transition metal catalyst, into the chiral alcohol of general formula 4 which is then reacted under suitable conditions to form the chiral oxiran of formula 5. Methods of synthesising oxirans from derivatives of the compound of formula 3 are known in the art (cf. for example Hamada et al., Org. Letters 2002, 4, 4373-4376).

By reacting the oxirans 5 with the amines of formula 6 the compounds of formula 7 are obtained, which may be converted into the compounds of formula 1 after cleaving of the protective group (PG). If the compounds of formula 1 are not obtained in crystalline form by the method of synthesis outlined above, the synthesis may be followed by recrystallisation from suitable solvents. More detailed comments on this subject can be found in the experimental part of the present invention that follows.

In order to prepare the inhalable powders according to the invention, it is first of all necessary to prepare the compounds of formula 1 obtained in crystalline form in finely-divided (or micronised) form.

The micronising or grinding process may be carried out using conventional mills. Preferably, the micronisation is carried out with the exclusion of moisture, more preferably, using a corresponding inert gas such as nitrogen, for example. It has proved particularly preferable to use air jet mills in which the material is comminuted by the impact of the particles on one another and on the walls of the grinding container. The micronising process may be carried out both by so-called counterflow mills, optionally with subsequent screening, and also, in preferred manner, using spiral air jet mills. According to the invention, nitrogen is preferably used as the grinding gas. The material for grinding is conveyed by the grinding gas under specific pressures (grinding pressure). Within the scope of the present invention, the grinding pressure is usually set to a value between about 2 and 8 bar, preferably between about 3 and 7 bar, most preferably between about 3.5 and 6.5 bar. The material for grinding is fed into the air jet mill by means of the feed gas under specific pressures (feed pressure). Within the scope of the present invention a feed pressure of between about 2 and 8 bar, preferably between about 3 and 7 bar and most preferably between about 3.5 and 6 bar has proved satisfactory. The feed gas used is also preferably an inert gas, most preferably nitrogen again. The material to be ground (crystalline compounds according to formula 1) may be fed in at a rate of about 5-45 g/min, preferably at about 15-35 g/min.

For example, without restricting the subject of the invention thereto, the following apparatus has proved suitable as a possible embodiment of an air jet mill: a 2-inch Microniser with grinding ring, 0.8 mm bore, made by Messrs Sturtevant Inc., 348 Circuit Street, Hanover, Mass. 02239, USA. Using this apparatus, the grinding process is preferably carried out with the following grinding parameters: grinding pressure: about 4.5-6.5 bar; feed pressure: about 4.5-6.5 bar; supply of grinding material: about 17-21 g/min.

Another example is the use of an air jet mill as made by Messrs Jetpharma, type Jetmill MC 50, which may be operated with the following process parameters:

| | |
|---|---|
| grinding pressure: | 8.0 bar (+/− 0.5 bar) |
| feed pressure: | 8.5 bar (+/− 0.5 bar), |
| | Note: the feed pressure is |
| | always set 0.25 to 0.5 bar |
| | higher than the grinding pressure |
| product feed: | 20 g/min (+/− 2.0 g/min) |
| nozzle setting | 37.2 mm (constant) |
| (injector): | |

The ground material thus obtained is then further processed under the following specific conditions. The micronisate is exposed to water vapour at a relative humidity of at least 40% at a temperature of 15-50° C., preferably 20-45° C., most preferably 25-40° C. Preferably, the humidity is set to a value of 50-95% r.h., preferably 60-90% r.h., most preferably 70-85% r.h. By relative humidity (r.h.) is meant within the scope of the present invention the quotient of the partial steam pressure and the steam pressure of the water at the temperature in question. Preferably, the micronisate obtained from the grinding process described above is subjected to the chamber conditions mentioned above for a period of at least 6 hours. Preferably, however, the micronisate is subjected to the chamber conditions mentioned above for about 12 to about 120 hours, preferably about 15 to about 96 hours, particularly preferably about 18 to about 72 hours. In an alternative embodiment this step is followed by drying. The ground material is exposed to an elevated temperature. For this, the micronised material is exposed to an elevated temperature of at least 40° C., preferably at least 50° C. and at most 70° C. for a period of at least 0.5 hours, preferably 0.5 hours to 6 hours, particularly preferably from 0.5 hours to 3 hours, at reduced relative humidity, i.e. a relative humidity of less than 60%, preferably less than 40% and particularly preferably less than 30%.

The micronised compounds of formula 1 according to the invention which may be obtained by the method described above have a characteristic particle size X₅₀ of between 0.1 μm and 10 μm, preferably between 0.5 μm and 6 μm, particularly preferably between 1.0 μm and 3.5 μm. In addition they are characterised by the parameter Q_{(5.8)} of more than 60%, preferably more than 70%, particularly preferably more than 80%.

The characteristic value X₅₀ denotes the median value of the particle size below which 50% of the quantity of particles are found, based on the volume distribution of the individual particles. The characteristic value Q_{(5.8)} indicates the quantity of particles below 5.8 μm, based on the volume distribution of the particles. The particle sizes were determined within the scope of the present invention by laser diffraction (Fraunhofer diffraction). The determination of the particle sizes by laser diffraction (Fraunhofer diffraction) was effected using the method described in WO 03/078429 (page 16 ff).

The micronised compounds of general formula 1 described hereinbefore may optionally be used for inhalation without any other excipient. Preferably, however, the pharmaceutical compositions according to the invention contain in addition to one or more, preferably one, compound of formula 1 at least one physiologically acceptable excipient or a mixture of physiologically acceptable excipients.

Examples of physiologically acceptable excipients which may be used to prepare the inhalable powders according to the invention include, for example, monosaccharides (e.g. glucose, fructose or arabinose), disaccharides (e.g. lactose, saccharose, maltose or trehalose), oligo- and polysaccharides (e.g. maltodextrin, starch, cellulose and the derivatives thereof), polylactide/glycolide (resomer), polyalcohols (e.g. sorbitol, mannitol, xylitol), amino acids (arginine hydrochloride), chitosan (particularly preferably lactose, mannitol, saccharose, sorbitol, trehalose), alkali metal and alkaline earth metal salts of stearic acid (e.g. Mg stearate), salts (e.g. sodium chloride, calcium carbonate) or mixtures of these excipients with one another. Preferably, mono- or disaccharides or polyalcohols are used, while the use of lactose, glucose, trehalose or mannitol, preferably lactose, mannitol or glucose, is preferred, particularly, but not exclusively, in the form of their hydrates. For the purposes of the invention, lactose or mannitol is the particularly preferred excipient, while lactose monohydrate or mannitol is most particularly preferred.

In pharmaceutical formulations according to the invention which contain in addition to a compound of formula 1 a physiologically acceptable excipient, the ratio of the compound of formula 1 to the excipient is usually kept in the range from 5:100 to 1:100000, preferably 3:1000 to 1:10000 and particularly preferably from 1:1000 to 3:10000, the ratios given above being ratios by weight (w/w).

The inhalable powders according to the invention are usually administered in amounts of 3-100 mg, preferably 5-50 mg for each inhalation.

If the inhalable powders according to the invention do not contain any excipient, only one or more, preferably one compound of formula 1 in micronised form, 1 to 30 μg, preferably 3 to 25 μg and particularly preferably 5 to 20 μg inhalable powder are usually administered per inhalation.

The inhalable powders according to the invention are preferably administered in the form of a pre-metered pharmaceutical preparation. Examples include an inhalation capsule system. It is also possible to use systems wherein the powder preparation is presented in single doses e.g. contained in blister wells. In another form the preparations according to the invention are also suitable for use in inhalers which have a powder reservoir and wherein the quantity of powder to be administered or the crystalline micronisate of the active substance is not metered or divided up until immediately prior to use. The powder preparations described here may be inhaled by means of a suitable inhaler. Suitable inhalers are known from the prior art. Particularly suitable inhalers are mentioned for example in WO 03/084502, the contents of which are hereby incorporated by reference with regard to the inhalers disclosed therein.

The inhalable powders prepared according to the invention may be prepared as described below.

The process according to the invention for preparing inhalable powders is characterised in that N+m substantially equal portions of the physiologically acceptable excipient and N equal portions of the micronised compound of formula 1 are placed in alternate layers in a suitable mixing vessel and after they have all been added the 2N+m layers of the two components are mixed together using a suitable mixer, a portion of the physiologically acceptable excipient being put in first, while N is an integer >0, preferably >1, and m denotes 0 or 1.

Preferably, the individual fractions are added in layers through a suitable screening apparatus. If desired, once the mixing process is finished, the entire powder mixture can be subjected to one or more additional screening processes. In the process according to the invention, N is naturally dependent inter alia on the total quantity of powder mixture to be produced. When producing smaller batches, the desired effect of high homogeneity in the sense of uniformity of content can be achieved with a smaller N. In principle, however, it is preferable according to the invention if N is at least 10 or more, more preferably 20 or more, better still 30 or more. The greater N is and, as a result, the greater the total number of layers of the powder fractions formed, the more homogeneous the powder mixture becomes in the sense of uniformity of content.

The number m may represent 0 or 1 within the scope of the process according to the invention. If m denotes 0 the last fraction added to the mixing apparatus, preferably screened into it, in a layer is the last portion of the micronised compound of formula 1. If m represents the number 1, the last fraction added to the mixing apparatus, preferably screened into it, in a layer is the last portion of the physiologically acceptable excipient. This may prove advantageous inasmuch as, when m=1, any residues of the last fraction of the active substance still remaining in the screening unit can be carried into the mixing unit by means of the last portion of excipient.

Preferably, the first portion of the N+m portions of the excipient is put in first, and then the first portion of the N portions of the active substance is placed in the mixing container. Whereas within the scope of the process according to the invention the individual components are normally added in roughly equal portions, it may be advantageous in some cases if the first of the N+m portions of excipient which is put into the mixing apparatus has a larger volume than the subsequent portions of excipient.

The inhalable powders according to the invention may also be prepared by first of all producing a mixture of active substance and excipient according to the method described above and then mixing the mixture thus obtained with more excipient. This may be done using the method described above, by mixing N batches of the active substance/excipient mixture layer by layer with N+m batches of other excipient.

The excipient used in the inhalable powders according to the invention preferably has an average particle size of 17-120 μm, preferably about 17-90 μm, particularly preferably about 20-60 μm. The excipient may optionally also be a mixture of coarser excipient with an average particle size of 17 to 75 μm and finer excipient with an average particle size of 1 to 9 μm, wherein the proportion of finer excipient in the total quantity of excipient may be 1 to 20%. If the inhalable powders which may be produced using the process according to the invention contain a mixture of coarser and finer excipient fractions, it is preferable according to the invention to prepare inhalable powders wherein the coarser excipient has an average particle size of 17 to 50 μm, most preferably 20 to 30 μm, and the finer excipient has an average particle size of 2 to 8 μm, most preferably 3 to 7 μm. By average particle size is meant here the 50% value of the volume distribution measured with a laser diffractometer using the dry dispersion method. For the measurement of the mean particle size by this method see the disclosure of WO 03/078429 (page 21 ff).

In the case of an excipient mixture of coarser and finer excipient fractions, the preferred processes according to the invention are those that produce inhalable powders in which the proportion of finer excipient constitutes 3 to 15%, most preferably 5 to 10% of the total amount of excipient.

The percentages given within the scope of the present invention are always percent by weight.

If the excipient used is one of the abovementioned mixtures of coarser excipient and finer excipient, it is again expedient according to the invention to produce the excipient mixture using the process according to the invention from N roughly equal portions of the finer excipient fraction with N+m roughly equal portions of the coarser excipient fraction. In such a case it is advisable first to generate the above-mentioned excipient mixture from the above-mentioned excipient fractions, and then to produce from it the total mixture including the active substance using the process according to the invention.

For example, the excipient mixture may be obtained as follows, using the process according to the invention. The two components are preferably added through a screening granulator with a mesh size of 0.1 to 2 mm, most preferably 0.3 to 1 mm, even more preferably 0.3 to 0.6 mm. Preferably the first fraction of the N+m portions of the coarser excipient is put in first and then the first portion of the N portions of the finer excipient fraction is added to the mixing container. The two components are added alternately by screening them in layer by layer.

After the preparation of the excipient mixture, the inhalable powder is produced from the mixture and the desired active substance using the process according to the invention. The two components are preferably added through a screening granulator with a mesh size of 0.1 to 2 mm, most preferably 0.3 to 1 mm, even more preferably 0.3 to 0.6 mm.

Preferably, the first portion of the N+m portions of the excipient mixture is put in and then the first portion of the N portions of the active substance is added to the mixing container. The two components are preferably added through a screening unit in alternate layers, in more than 20, preferably more than 25, most preferably more than 30 layers. For example, with a desired total amount of powder of 30-35 kg containing 0.3-0.5% of active substance, for example, and using common excipients, the two components can be screened in in about 30 to 60 layers each (N=30-60). As will be clearly apparent to anyone skilled in the art, the process can equally well be carried out with N>60 to achieve the desired effect of the maximum possible homogeneity of the powder mixture.

The inhalable powders according to the invention are characterised by their multiplicity of possible applications in the therapeutic field. Particular mention should be made according to the invention of those applications for which the compounds of formula 1 according to the invention are preferably used on the basis of their pharmaceutical activity as betamimetics.

Accordingly, in another aspect, the present invention relates to the above-mentioned inhalable powders as pharmaceutical compositions. The present invention also relates to the use of the above-mentioned inhalable powders for preparing a pharmaceutical composition for the treatment of respiratory complaints.

The present invention preferably relates to the use of the above-mentioned inhalable powders for preparing a pharmaceutical composition for the treatment of respiratory complaints selected from the group comprising obstructive pulmonary diseases of various origins, pulmonary emphysema of various origins, restrictive pulmonary diseases, interstitial pulmonary diseases, cystic fibrosis, bronchitis of various origins, bronchiectasis, ARDS (adult respiratory distress syndrome) and all forms of pulmonary oedema.

Preferably, the inhalable powders according to the invention are used to prepare a pharmaceutical composition for the treatment of obstructive pulmonary diseases selected from the group consisting of COPD (chronic obstructive pulmonary disease), bronchial asthma, paediatric asthma, severe asthma, acute asthma attacks and chronic bronchitis, while their use for preparing a pharmaceutical composition for the treatment of bronchial asthma is particularly preferred according to the invention.

Also preferably, the inhalable powders according to the invention are used to prepare a pharmaceutical composition for the treatment of pulmonary emphysema which has its origins in COPD (chronic obstructive pulmonary disease) or α1-proteinase inhibitor deficiency.

Also preferably, the inhalable powders according to the invention are used to prepare a pharmaceutical composition for the treatment of restrictive pulmonary diseases selected from among allergic alveolitis, restrictive pulmonary diseases triggered by work-related noxious substances, such as asbestosis or silicosis, and restriction caused by lung tumours, such as for example lymphangiosis carcinomatosa, bronchoalveolar carcinoma and lymphomas.

Also preferably, the inhalable powders according to the invention are used to prepare a pharmaceutical composition for the treatment of interstitial pulmonary diseases selected from among pneumonia caused by infections, such as for example infection by viruses, bacteria, fungi, protozoa, helminths or other pathogens, pneumonitis caused by various factors, such as for example aspiration and left heart insufficiency, radiation-induced pneumonitis or fibrosis, collagenoses, such as for example lupus erythematodes, systemic sclerodermy or sarcoidosis, granulomatoses, such as for example Boeck's disease, idiopathic interstitial pneumonia or idiopathic pulmonary fibrosis (IPF).

Also preferably, the inhalable powders according to the invention are used to prepare a pharmaceutical composition for the treatment of cystic fibrosis or mucoviscidosis.

Also preferably, the inhalable powders according to the invention are used to prepare a pharmaceutical composition for the treatment of bronchitis, such as for example bronchitis caused by bacterial or viral infection, allergic bronchitis and toxic bronchitis.

Also preferably, the inhalable powders according to the invention are used to prepare a pharmaceutical composition for the treatment of bronchiectasis.

Also preferably, the inhalable powders according to the invention are used to prepare a pharmaceutical composition for the treatment of ARDS (adult respiratory distress syndrome).

Also preferably, the inhalable powders according to the invention are used to prepare a pharmaceutical composition for the treatment pulmonary oedema, for example toxic pulmonary oedema after aspiration or inhalation of toxic substances and foreign substances.

Particularly preferably the present invention relates to the use of the inhalable powders according to the invention for preparing a pharmaceutical composition for the treatment of asthma or COPD. Also of particular importance is the above-mentioned use of the inhalable powders according to the invention for preparing a pharmaceutical composition for once-a-day treatment of inflammatory and obstructive respiratory complaints, particularly for the once-a-day treatment of asthma or COPD.

The present invention also relates to a process for the treatment of the above-mentioned diseases, characterised in that one or more of the above-mentioned inhalable powders according to the invention are administered in therapeutically effective amounts. The present invention preferably also relates to processes for the treatment of asthma or COPD, characterised in that one or more of the above-mentioned inhalable powders according to the invention are administered once a day in therapeutically effective amounts.

The examples of synthesis described below serve to illustrate the invention in more detail. However, they are intended only as examples of procedures to illustrate the invention without restricting it to the subject matter described in an exemplifying capacity hereinafter.

Preparation of the Compounds of General Formula 1:

### EXAMPLE 1

### 6-hydroxy-8-{(R)-1-hydroxy-2-[2-(4-methoxy-phenyl)-1,1-dimethyl-ethylamino]-ethyl}-4H-benzo[1,4]oxazin-3-one-hydrochloride

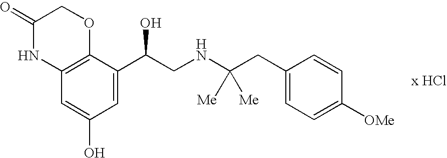

### a) 1-(5-benzyloxy-2-hydroxy-3-nitro-phenyl)-ethanone

18 mL of fuming nitric acid are added dropwise to a solution of 81.5 g (0.34 mol) 1-(5-benzyloxy-2-hydroxy-phenyl)-ethanone in 700 mL acetic acid, while being cooled with the ice bath, in such a way that the temperature does not exceed 20° C. Then the reaction mixture is stirred for two hours at ambient temperature, poured onto ice water and filtered. The product is recrystallised from isopropanol, suction filtered and washed with isopropanol and diisopropylether.

Yield: 69.6 g (72%); mass spectroscopy [M+H]⁺=288.

### b) 1-(3-amino-5-benzyloxy-2-hydroxy-phenyl)-ethanone

69.5 g (242 mmol) 1-(5-benzyloxy-2-hydroxy-3-nitro-phenyl)-ethanone are dissolved in 1.4 L methanol and hydrogenated in the presence of 14 g rhodium on charcoal (10%) as catalyst at 3 bar and ambient temperature. Then the catalyst is filtered off and the filtrate is evaporated down. The residue is reacted further without any additional purification.

Yield: 60.0 g (96%), R_{f} value=0.45 (dichloromethane on silica gel).

### c) 8-acetyl-6-benzyloxy-4H-benzo[1,4]oxazin-3-one

21.0 mL (258 mmol) chloroacetyl chloride are added dropwise to 60.0 g (233 mmol) 1-(3-amino-5-benzyloxy-2-hydroxy-phenyl)-ethanone and 70.0 g (506 mmol) potassium carbonate while being cooled with the ice bath. Then the mixture is stirred overnight at ambient temperature and then for 6 hours at reflux temperature. The hot reaction mixture is filtered, then evaporated down to approx. 400 mL and combined with ice water. The precipitate formed was suction filtered, dried and purified by chromatography on a short silica gel column (dichloromethane:methanol=99:1). The fractions containing the product are evaporated down, suspended in isopropanol/diisopropylether, suction filtered and washed with diisopropylether.

Yield: 34.6 g (50%); mass spectroscopy [M+H]⁺=298.

### d) 6-benzyloxy-8-(2-chloro-acetyl)-4H-benzo[1,4]oxazin-3-one

13.8 g (46.0 mmol) 8-acetyl-6-benzyloxy-4H-benzo[1,4]oxazin-3-one and 35.3 g (101.5 mmol) benzyltrimethylammonium-dichloriodate are stirred in 250 mL dichloroethane, 84 mL glacial acetic acid and 14 mL water for 5 hours at 65° C. After cooling to ambient temperature 5% sodium hydrogen sulphite solution is added and the mixture is stirred for 30 minutes. The precipitated solid is suction filtered, washed with water and diethyl ether and dried.

Yield: 13.2 g (86%); mass spectroscopy [M+H]⁺=330/32.

### e) 6-benzyloxy-8-((R)-2-chloro-1-hydroxy-ethyl)-4H-benzo[1,4]-oxazin-3-one

The process is carried out analogously to a process described in the literature (Org. Lett. 2002, 4, 4373-4376).

8 mL of a mixture of formic acid and triethylamine (molar ratio=5:2) are added dropwise at −15° C. to 13.15 g (39.6 mmol) 6-benzyloxy-8-(2-chloro-acetyl)-4H-benzo[1,4]oxazin-3-one and 25.5 mg (0.04 mmol) Cp*RhCl[(S,S)-TsDPEN] (Cp*=pentamethylcyclopentadienyl and TsDPEN=(1S,2S)-N-p-toluenesulphonyl-1,2-diphenylethylenediamine) in 40 mL dimethylformamide. The mixture is stirred for 5 hours at this temperature, then 25 mg catalyst are added and the mixture is stirred overnight at −15° C. The reaction mixture is combined with ice water and filtered. The filter residue is dissolved in dichloromethane, dried with sodium sulphate and freed from the solvent. The residue is chromatographed (dichloromethane/methanol-gradient) and the product is recrystallised from diethyl ether/diisopropylether.

Yield: 10.08 g (76%); R_{f} value=0.28 (dichloromethane:methanol=50:1 on silica gel).

### f) 6-benzyloxy-8-(R)-oxiranyl-4H-benzo[1,4]oxazin-3-one

10.06 g (30.1 mmol) 6-benzyloxy-8-((R)-2-chloro-1-hydroxy-ethyl)-4H-benzo[1,4]-oxazin-3-one are dissolved in 200 mL dimethylformamide. The solution is combined with 40 mL of a 2 molar sodium hydroxide solution at 0° C. and stirred for 4 hours at this temperature. The reaction mixture is poured onto ice water, stirred for 15 minutes and then filtered. The solid is washed with water and dried. Yield: 8.60 g (96%); mass spectroscopy [M+H]⁺=298.

### g) 6-benzyloxy-8-{(R)-1-hydroxy-2-[2-(4-methoxy-phenyl)-1,1-dimethyl-ethylamino]-ethyl}-4H-benzo[1,4,]oxazin-3-one

5.25 g (17.7 mmol) 6-benzyloxy-8-(R)-oxiranyl-4H-benzo[1,4]oxazin-3-one and 6.30 g (35.1 mmol) 2-(4-methoxy-phenyl)-1,1-dimethyl-ethylamine are combined with 21 mL isopropanol and stirred for 30 minutes at 135° C. under microwave radiation in a sealed reaction vessel. The solvent is distilled off and the residue is chromatographed (aluminium oxide; ethyl acetate/methanol gradient). The product thus obtained is purified by recrystallisation from a diethyl ether/diisopropylether mixture. Yield: 5.33 g (63%); mass spectroscopy [M+H]⁺=477.

### h) 6-hydroxy-8-{(R)-1-hydroxy-2-[2-(4-methoxy-phenyl)-1,1-dimethyl-ethylamino]-ethyl}-4H-benzo[1,4,]oxazin-3-one-hydrochloride

A suspension of 5.33 g (11.2 mmol) 6-benzyloxy-8-{(R)-1-hydroxy-2-[2-(4-methoxy-phenyl)-1,1-dimethyl-ethylamino]-ethyl}-4H-benzo[1,4]oxazin-3-one in 120 mL methanol is combined with 0.8 g palladium on charcoal (10%), heated to 50° C. heated and hydrogenated at 3 bar hydrogen pressure. Then the catalyst is suction filtered and the filtrate is evaporated down. The residue is dissolved in 20 mL isopropanol and 2.5 mL of 5 molar hydrochloric acid in isopropanol are added. The product is precipitated with 200 mL diethyl ether, suction filtered and dried. Yield: 4.50 g (95%, hydrochloride); mass spectroscopy [M+H]⁺=387.

The following compounds of formula 1 are obtained analogously by reacting the compound 6-benzyloxy-8-(R)-oxiranyl-4H-benzo[1,4]oxazin-3-one (Example 1, Step f) with the corresponding amine.

### EXAMPLE 2

### 8-{(R)-2-[2-(2,4-difluoro-phenyl)-1,1-dimethyl-ethylamino]-1-hydroxy-ethyl}-6-hydroxy-4H-benzo[1,4]oxazin-3-one-hydrochloride

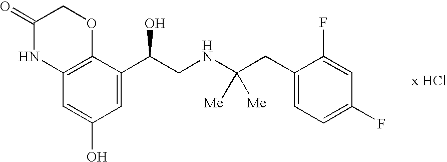

Mass spectroscopy [M+H]⁺=393.

### EXAMPLE 3

### 8-{(R)-2-[2-(3,5-difluoro-phenyl)-1,1-dimethyl-ethylamino]-1-hydroxy-ethyl}-6-hydroxy-4H-benzo[1,4]oxazin-3-one-hydrochloride

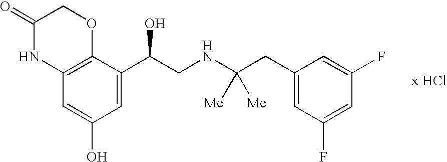

Mass spectroscopy [M+H]⁺=393.

### EXAMPLE 4

### 8-{(R)-2-[2-(4-ethoxy-phenyl)-1,1-dimethyl-ethylamino]-1-hydroxy-ethyl}-6-hydroxy-4H-benzo[1,4]oxazin-3-one-hydrochloride

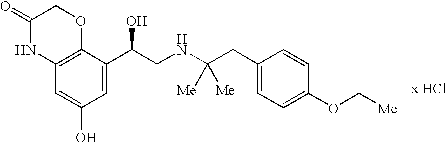

Mass spectroscopy [M+H]⁺=401.

### EXAMPLE 5

### 8-{(R)-2-[2-(4-fluoro-phenyl)-1,1-dimethyl-ethylamino]-1-hydroxy-ethyl}-6-hydroxy-4H-benzo[1,4]oxazin-3-one-hydrochloride

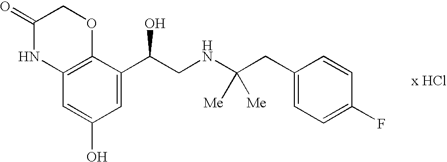

Mass spectroscopy [M+H]⁺=375.

If the compounds of formula 1 according to the method of synthesis described above by way of example do not lead to uniform crystal modifications, it may be useful to recrystallise the salts of formula 1 obtained from suitable solvents. Moreover, other salts may be obtained from the above-mentioned Examples using methods known per se from the prior art.

In the next section, methods of preparing uniform salts of the compounds of formula 1 which are particularly suitable for preparing formulations for administration by inhalation are described by way of example.

### EXAMPLE 6

### 6-hydroxy-8-{(R)-1-hydroxy-2-[2-(4-methoxy-phenyl)-1,1-dimethyl-ethylamino]-ethyl}-4H-benzo[1,4,]oxazin-3-one maleate

250 mg (0.65 mmol) 6-hydroxy-8-{(R)-1-hydroxy-2-[2-(4-methoxy-phenyl)-1,1-dimethyl-ethylamino]-ethyl}-4H-benzo[1,4,]oxazin-3-one are combined with sufficient ethanol to make the solid go into solution completely. Then 75 mg (0.65 mmol) maleic acid and a crystallisation aid are added. The mixture is cooled with ice and the precipitated solid is filtered off and washed with ethanol and diethyl ether. In the salt the acid and the ethanolamine are present in the ratio 1:1.

Yield: 254 mg (78%); mass spectroscopy [M+H]⁺=387; melting point=215° C.

The highly crystalline product was investigated further by X-ray powder diffraction. The X-ray powder diagram was recorded using the following method.

The X-ray powder diagram was recorded within the scope of the present invention using a Bruker D8 Advanced with an LSD (=location-sensitive detector) (CuK_{α} radiation, λ=1.5418 Å, 30 kV, 40 mA).

For the highly crystalline compound the following characteristic values dₕₖₗ [Å] were obtained, inter alia, which give the lattice plane intervals determined in Å:
d=21.68 Å; 8.62 Å; 5.92 Å; 5.01 Å; 4.59 Å; 4.36 Å; 3.64 Å and 3.52 Å.

### EXAMPLE 7

### 6-hydroxy-8-{(R)-1-hydroxy-2-[2-(4-methoxy-phenyl)-1,1-dimethyl-ethylamino]-ethyl}-4H-benzo[1,4,]oxazin-3-one salicylate

250 mg (0.65 mmol) 6-hydroxy-8-{(R)-1-hydroxy-2-[2-(4-methoxy-phenyl)-1,1-dimethyl-ethylamino]-ethyl}-4H-benzo[1,4,]oxazin-3-one are dissolved in a little ethanol and combined with 90 mg (0.65 mmol) salicylic acid. After the addition of a crystallisation aid the mixture is left to stand overnight, during which time a solid is precipitated. Diethyl ether is added and after 30 minutes the mixture is filtered. The white solid thus obtained is washed with diethyl ether and dried.

Yield: 295 mg (87%); mass spectroscopy [M+H]⁺=387; melting point=215° C.

The highly crystalline product was investigated further by X-ray powder diffraction. The X-ray powder diagram was recorded using the following method.

The X-ray powder diagram was recorded within the scope of the present invention using a Bruker D8 Advanced with an LSD (=location-sensitive detector) (CuK_{α} radiation, λ=1.5418 Å, 30 kV, 40 mA).

For the highly crystalline compound the following characteristic values dₕₖₗ [Å] were obtained, inter alia, which give the lattice plane intervals determined in Å:
d=9.06 Å; 8.36 Å; 8.02 Å; 6.84 Å; 6.73 Å; 4.48 Å; 4.35 Å and 4.27 Å.

### EXAMPLE 8

### 6-hydroxy-8-{(R)-1-hydroxy-2-[2-(4-methoxy-phenyl)-1,1-dimethyl-ethylamino]-ethyl}-4H-benzo[1,4,]oxazin-3-one succinate

500 mg (1.2 mmol) 6-hydroxy-8-{(R)-1-hydroxy-2-[2-(4-methoxy-phenyl)-1,1-dimethyl-ethylamino]-ethyl}-4H-benzo[1,4,]oxazin-3-one hydrochloride are combined with ethyl acetate and extracted with aqueous potassium carbonate solution, the organic phase is dried with sodium sulphate and freed from the solvent. The residue is dissolved in a little ethanol and combined with 140 mg (1.2 mmol) succinic acid. After 2 hours the precipitated solid is suction filtered and washed with cold ethanol and diethyl ether. The ethanolamine and acid are present in the salt in a ratio of 1 to 0.5.

Yield: 468 mg (85%); mass spectroscopy [M+H]⁺=387; melting point=115° C.

The highly crystalline product was investigated further by X-ray powder diffraction. The X-ray powder diagram was recorded using the following method.

The X-ray powder diagram was recorded within the scope of the present invention using a Bruker D8 Advanced with an LSD (=location-sensitive detector) (CuK_{α} radiation, λ=1.5418 Å, 30 kV, 40 mA).

For the highly crystalline compound the following characteristic values dₕₖₗ [Å] were obtained, inter alia, which give the lattice plane intervals determined in Å:
d=14.35 Å; 8.49 Å; 7.37 Å; 7.25 Å; 5.47 Å; 4.78 Å; 4.14 Å and 3.59 Å.

### EXAMPLE 9

### 6-hydroxy-8-{(R)-1-hydroxy-2-[2-(4-methoxy-phenyl)-1,1-dimethyl-ethylamino]-ethyl}-4H-benzo[1,4,]oxazin-3-one-fumarate

300 mg (0.71 mmol) 6-hydroxy-8-{(R)-1-hydroxy-2-[2-(4-methoxy-phenyl)-1,1-dimethyl-ethylamino]-ethyl}-4H-benzo[1,4,]oxazin-3-one hydrochloride are combined with ethyl acetate and extracted with aqueous potassium carbonate solution. The organic phase is dried with sodium sulphate and freed from the solvent. The residue is dissolved in ethanol with the addition of a few drops of water. 82 mg (0.71 mmol) fumaric acid and seed crystals are added and the mixture is left to stand overnight. The white solid is suction filtered, washed with diethyl ether and ethanol and dried. The ethanolamine and acid are present in the salt in a ratio of 1 to 0.5.

Yield: 208 mg (63%); mass spectroscopy [M+H]⁺=387; melting point=130° C.

The highly crystalline product was investigated further by X-ray powder diffraction. The X-ray powder diagram was recorded using the following method.

The X-ray powder diagram was recorded within the scope of the present invention using a Bruker D8 Advanced with an LSD (=location-sensitive detector) (CuK_{α} radiation, λ=1.5418 Å, 30 kV, 40 mA).

For the highly crystalline compound the following characteristic values dₕₖₗ [Å] were obtained, inter alia, which give the lattice plane intervals determined in Å:
d=14.23 Å; 5.44 Å; 4.76 Å; 4.57 Å; 4.26 Å; 4.12 Å; 3.57 Å and 3.48 Å.

### EXAMPLE 10

### 6-hydroxy-8-{1-hydroxy-2-[2-(4-methoxy-phenyl)-1,1-dimethyl-ethylamino]-ethyl}-4H-benzo[1,4,]oxazin-3-one (free base)

Analogously to the preceding tests, 500 mg (1.2 mmol) 6-hydroxy-8-{(R)-1-hydroxy-2-[2-(4-methoxy-phenyl)-1,1-dimethyl-ethylamino]-ethyl}-4H-benzo[1,4,]oxazin-3-one hydrochloride are first of all combined with ethyl acetate. The organic phase is extracted with aqueous potassium carbonate solution, dried with sodium sulphate and freed from the solvent. The free base thus obtained is dissolved in acetonitrile with the addition of a few drops of water. The precipitated solid is suction filtered, washed and dried.

Yield: 168 mg (37%); mass spectroscopy [M+H]⁺=387; melting point=128° C.

The highly crystalline product was investigated further by X-ray powder diffraction. The X-ray powder diagram was recorded using the following method.

The X-ray powder diagram was recorded within the scope of the present invention using a Bruker D8 Advanced with an LSD (=location-sensitive detector) (CuK_{α} radiation, λ=1.5418 Å, 30 kV, 40 mA).

For the highly crystalline compound the following characteristic values dₕₖₗ [Å] were obtained, inter alia, which give the lattice plane intervals determined in Å:
d=14.96 Å; 9.63 Å; 7.05 Å; 5.57 Å; 5.28 Å; 5.05 Å; 4.63 Å and 3.73 Å.

### EXAMPLE 11

### 6-hydroxy-8-{(R)-1-hydroxy-2-[2-(4-methoxy-phenyl)-1,1-dimethyl-ethylamino]-ethyl}-4H-benzo[4,1,]oxazin-3-one-hydrochloride

300 mg (0.71 mmol) 6-hydroxy-8-{(R)-1-hydroxy-2-[2-(4-methoxy-phenyl)-1,1-dimethyl-ethylamino]-ethyl}-4H-benzo[1,4,]oxazin-3-one hydrochloride are dissolved by heating in 4 mL isopropanol. The solution is cooled to ambient temperature and then placed in an ice bath for 15 minutes. The precipitated solid is suction filtered and dried.

Yield: 180 mg (60%); mass spectroscopy [M+H]⁺=387; melting point=211° C.

The highly crystalline product was investigated further by X-ray powder diffraction. The X-ray powder diagram was recorded using the following method.

The X-ray powder diagram was recorded within the scope of the present invention using a Bruker D8 Advanced with an LSD (=location-sensitive detector) (CuK_{α} radiation, λ=1.5418 Å, 30 kV, 40 mA).

For the highly crystalline compound the following characteristic values dₕₖₗ [Å] were obtained, inter alia, which give the lattice plane intervals determined in Å:
d=5.92 Å; 5.81 Å; 5.51 Å; 5.10 Å; 4.65 Å; 4.50 Å; 4.15 Å and 4.00 Å.

The following compounds may be obtained analogously using the method described in Examples 6 to 11:
Example 12: 8-{(R)-2-[2-(2,4-difluoro-phenyl)-1,1-dimethyl-ethylamino]-1-hydroxy-ethyl}-6-hydroxy-4H-benzo[1,4]oxazin-3-one-maleate;
Example 13: 8-{(R)-2-[2-(2,4-difluoro-phenyl)-1,1-dimethyl-ethylamino]-1-hydroxy-ethyl}-6-hydroxy-4H-benzo[1,4]oxazin-3-one-salicylate;
Example 14: 8-{(R)-2-[2-(2,4-difluoro-phenyl)-1,1-dimethyl-ethylamino]-1-hydroxy-ethyl}-6-hydroxy-4H-benzo[1,4]oxazin-3-one-succinate;
Example 15: 8-{(R)-2-[2-(2,4-difluoro-phenyl)-1,1-dimethyl-ethylamino]-1-hydroxy-ethyl}-6-hydroxy-4H-benzo[1,4]oxazin-3-one-fumarate;
Example 16: 8-{(R)-2-[2-(3,5-difluoro-phenyl)-1,1-dimethyl-ethylamino]-1-hydroxy-ethyl}-6-hydroxy-4H-benzo[1,4]oxazin-3-one-maleate;
Example 17: 8-{(R)-2-[2-(3,5-difluoro-phenyl)-1,1-dimethyl-ethylamino]-1-hydroxy-ethyl}-6-hydroxy-4H-benzo[1,4]oxazin-3-one-salicylate;
Example 18: 8-{(R)-2-[2-(3,5-difluoro-phenyl)-1,1-dimethyl-ethylamino]-1-hydroxy-ethyl}-6-hydroxy-4H-benzo[1,4]oxazin-3-one-succinate;
Example 19: 8-{(R)-2-[2-(3,5-difluoro-phenyl)-1,1-dimethyl-ethylamino]-1-hydroxy-ethyl}-6-hydroxy-4H-benzo[1,4]oxazin-3-one-fumarate;
Example 20: 8-{(R)-2-[2-(4-ethoxy-phenyl)-1,1-dimethyl-ethylamino]-1-hydroxy-ethyl}-6-hydroxy-4H-benzo[1,4]oxazin-3-one-maleate;
Example 21: 8-{(R)-2-[2-(4-ethoxy-phenyl)-1,1-dimethyl-ethylamino]-1-hydroxy-ethyl}-6-hydroxy-4H-benzo[1,4]oxazin-3-one-salicylate;
Example 22: 8-{(R)-2-[2-(4-ethoxy-phenyl)-1,1-dimethyl-ethylamino]-1-hydroxy-ethyl}-6-hydroxy-4H-benzo[1,4]oxazin-3-one-succinate;
Example 23: 8-{(R)-2-[2-(4-ethoxy-phenyl)-1,1-dimethyl-ethylamino]-1-hydroxy-ethyl}-6-hydroxy-4H-benzo[1,4]oxazin-3-one-fumarate;
Example 24: 8-{(R)-2-[2-(4-fluoro-phenyl)-1,1-dimethyl-ethylamino]-1-hydroxy-ethyl}-6-hydroxy-4H-benzo[1,4]oxazin-3-one-maleate;
Example 25: 8-{(R)-2-[2-(4-fluoro-phenyl)-1,1-dimethyl-ethylamino]-1-hydroxy-ethyl}-6-hydroxy-4H-benzo[1,4]oxazin-3-one-salicylate;
Example 26: 8-{(R)-2-[2-(4-fluoro-phenyl)-1,1-dimethyl-ethylamino]-1-hydroxy-ethyl}-6-hydroxy-4H-benzo[1,4]oxazin-3-one-succinate;
Example 27: 8-{(R)-2-[2-(4-fluoro-phenyl)-1,1-dimethyl-ethylamino]-1-hydroxy-ethyl}-6-hydroxy-4H-benzo[1,4]oxazin-3-one-fumarate;
Example 28: 8-{(R)-2-[2-(2,4-difluoro-phenyl)-1,1-dimethyl-ethylamino]-1-hydroxy-ethyl}-6-hydroxy-4H-benzo[1,4]oxazin-3-one (free base);
Example 29: 8-{(R)-2-[2-(3,5-difluoro-phenyl)-1,1-dimethyl-ethylamino]-1-hydroxy-ethyl}-6-hydroxy-4H-benzo[1,4]oxazin-3-one (free base);
Example 30: 8-{(R)-2-[2-(4-ethoxy-phenyl)-1,1-dimethyl-ethylamino]-1-hydroxy-ethyl}-6-hydroxy-4H-benzo[1,4]oxazin-3-one (free base) or also
Example 31: 8-{(R)-2-[2-(4-fluoro-phenyl)-1,1-dimethyl-ethylamino]-1-hydroxy-ethyl}-6-hydroxy-4H-benzo[1,4]oxazin-3-one (free base).
   Preparation of the Powder Formulations According to the Invention:
   I) Apparatus

The following machines and equipment may be used, for example, to prepare the inhalable powders:
Mixing container or powder mixer: Turbulamischer 2 L, type 2C; manufactured by Willy A. Bachofen AG, CH-4500 Basel
Hand-held screen: 0.315 mm mesh size

The empty inhalant capsules may be filled with inhalable powder containing the active substance manually or by machine. The following equipment may be used.
Capsule filling machine:
MG2, type G100, manufacturer: MG2 S.r.l, I-40065 Pian di Macina di Pianoro (BO), Italy

### EXAMPLE 1

Powder Mixture:

In order to prepare the powder mixture 97.0 g excipient (lactose monohydrate 200 mesh with an average particle size of 25-50 μm, which varies from one batch to another) and 3.0 g micronised compound of formula 1 are used. The proportion of active substance in the 100 g inhalable powder obtained is 3.0%.

The excipient is placed in a suitable mixing container through a hand-held screen with a mesh size of 0.315 mm. Then 3 g of micronised compound of formula 1 and 7 g of excipient are screened in in alternate layers. The excipient and the active substance are added in 7 and 6 layers, respectively (premix I). The constituents screened in are then mixed (mixing: 30 rpm/30 min).

10 g of premix I and 90 g excipient are then added in alternate layers through the same hand-held screen with a mesh size of 0.315 mm by screening into a suitable mixing container. The excipient and the premix I are added in 8-10 layers (final mix). The constituents screened in are then mixed (mixing: 30 rpm/30 min).

The following inhalable powders may be obtained according to or analogously to the procedure described in Example 1:

### EXAMPLE 2

| | | |
|---|---|---|
| active substance = Example 6 | 0.6 | g |
| Lactose monohydrate: | 99.4 | g |
| Total: | 100.0 | g |

### EXAMPLE 3

| | | |
|---|---|---|
| active substance = Example 6 | 3.0 | g |
| Lactose monohydrate: | 97.0 | g |
| Total: | 100.0 | g |

### EXAMPLE 4

| | | |
|---|---|---|
| active substance = Example 9 | 0.6 | g |
| Lactose monohydrate: | 99.4 | g |
| Total: | 100.0 | g |

### EXAMPLE 5

| | | |
|---|---|---|
| active substance = Example 10 | 0.6 | g |
| Lactose monohydrate: | 99.4 | g |
| Total: | 100.0 | g |

### EXAMPLE 6

| | | |
|---|---|---|
| active substance = Example 11 | 3.0 | g |
| Lactose monohydrate: | 97.0 | g |
| Total: | 100.0 | g |

### EXAMPLE 7

| | | |
|---|---|---|
| active substance = Example 13 | 0.6 | g |
| Lactose monohydrate: | 99.4 | g |
| Total: | 100.0 | g |

### EXAMPLE 8

| | | |
|---|---|---|
| active substance = Example 11 | 0.6 | g |
| Lactose monohydrate: | 99.4 | g |
| Total: | 100.0 | g |

### EXAMPLE 9

| | | |
|---|---|---|
| active substance = Example 10 | 3.0 | g |
| Lactose monohydrate: | 97.0 | g |
| Total: | 100.0 | g |

### EXAMPLE 10

| | | |
|---|---|---|
| active substance = Example 13 | 0.6 | g |
| Lactose monohydrate: | 99.4 | g |
| Total: | 100.0 | g |

### EXAMPLE 11

| | | |
|---|---|---|
| active substance = Example 14 | 0.6 | g |
| Lactose monohydrate: | 99.4 | g |
| Total: | 100.0 | g |

### EXAMPLE 12

| | | |
|---|---|---|
| active substance = Example 14 | 3.0 | g |
| Lactose monohydrate: | 97.0 | g |
| Total: | 100.0 | g |

### EXAMPLE 13

| | | |
|---|---|---|
| active substance = Example 15 | 0.6 | g |
| Lactose monohydrate: | 99.4 | g |
| Total: | 100.0 | g |

### EXAMPLE 14

| | | |
|---|---|---|
| active substance = Example 8 | 0.6 | g |
| Lactose monohydrate: | 99.4 | g |
| Total: | 100.0 | g |

### EXAMPLE 15

| | | |
|---|---|---|
| active substance = Example 8 | 3.0 | g |
| Lactose monohydrate: | 97.0 | g |
| Total: | 100.0 | g |

### EXAMPLE 16

| | | |
|---|---|---|
| active substance = Example 7 | 0.6 | g |
| Lactose monohydrate: | 99.4 | g |
| Total: | 100.0 | g |

### EXAMPLE 17

| | | |
|---|---|---|
| active substance = Example 6 | 0.6 | g |
| Mannitol: | 99.4 | g |
| Total: | 100.0 | g |

### EXAMPLE 18

| | | |
|---|---|---|
| active substance = Example 6 | 3.0 | g |
| Mannitol: | 97.0 | g |
| Total: | 100.0 | g |

### EXAMPLE 19

| | | |
|---|---|---|
| active substance = Example 9 | 0.6 | g |
| Mannitol | 99.4 | g |
| Total: | 100.0 | g |

### EXAMPLE 20

| | | |
|---|---|---|
| active substance = Example 10 | 0.6 | g |
| Mannitol: | 99.4 | g |
| Total: | 100.0 | g |

### EXAMPLE 21

| | | |
|---|---|---|
| active substance = Example 11 | 3.0 | g |
| Mannitol: | 97.0 | g |
| Total: | 100.0 | g |

### EXAMPLE 22

| | | |
|---|---|---|
| active substance = Example 13 | 0.6 | g |
| Mannitol: | 99.4 | g |
| Total: | 100.0 | g |

### EXAMPLE 23

| | | |
|---|---|---|
| active substance = Example 11 | 0.6 | g |
| Mannitol: | 99.4 | g |
| Total: | 100.0 | g |

### EXAMPLE 24

| | | |
|---|---|---|
| active substance = Example 10 | 3.0 | g |
| Mannitol: | 97.0 | g |
| Total: | 100.0 | g |

### EXAMPLE 25

| | | |
|---|---|---|
| active substance = Example 13 | 0.6 | g |
| Mannitol: | 99.4 | g |
| Total: | 100.0 | g |

### EXAMPLE 26

| | | |
|---|---|---|
| active substance = Example 14 | 0.6 | g |
| Mannitol: | 99.4 | g |
| Total: | 100.0 | g |

### EXAMPLE 27

| | | |
|---|---|---|
| active substance = Example 14 | 3.0 | g |
| Mannitol: | 97.0 | g |
| Total: | 100.0 | g |

### EXAMPLE 28

| | | |
|---|---|---|
| active substance = Example 15 | 0.6 | g |
| Mannitol: | 99.4 | g |
| Total: | 100.0 | g |

### EXAMPLE 29

| | | |
|---|---|---|
| active substance = Example 8 | 0.6 | g |
| Mannitol: | 99.4 | g |
| Total: | 100.0 | g |

### EXAMPLE 30

| | | |
|---|---|---|
| active substance = Example 8 | 3.0 | g |
| Mannitol: | 97.0 | g |
| Total: | 100.0 | g |

### EXAMPLE 31

| | | |
|---|---|---|
| active substance = Example 7 | 0.6 | g |
| Mannitol: | 99.4 | g |
| Total: | 100.0 | g |

1) An inhalable powder containing one or more enantiomerically pure compounds of formula 1 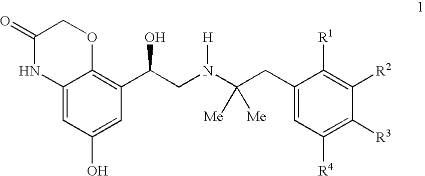
wherein
R¹ denotes hydrogen, C₁-C₄-alkyl, C₁-C₄-alkoxy or halogen;
R² denotes hydrogen, C₁-C₄-alkyl, C₁-C₄-alkoxy or halogen;
R³ denotes hydrogen, C₁-C₄-alkyl, C₁-C₄-alkoxy, halogen, OH, —O—C₁-C₄-alkylene-COOH or —O—C₁-C₄-alkylene-COO—C₁-C₄-alkyl;
R⁴ denotes hydrogen, C₁-C₄-alkyl, C₁-C₄-alkoxy or halogen,
optionally in the form of the pharmaceutically acceptable acid addition salts, hydrates or solvates thereof, optionally in admixture with one or more physiologically acceptable excipients.

2) An inhalable powder according to claim 1 which contains one or more enantiomerically pure compounds of formula 1, wherein
R¹ denotes hydrogen or halogen;
R² denotes hydrogen or halogen;
R³ denotes hydrogen, C₁-C₄-alkoxy or halogen;
R⁴ denotes hydrogen or halogen;
optionally in the form of the pharmaceutically acceptable acid addition salts, hydrates or solvates thereof, optionally in admixture with one or more physiologically acceptable excipients.

3) An inhalable powder according to claim 1 which contains one or more enantiomerically pure compounds of formula 1, wherein
R¹ denotes hydrogen, fluorine or chlorine, preferably hydrogen or fluorine;
R² denotes hydrogen, fluorine or chlorine, preferably hydrogen or fluorine;
R³ hydrogen, methoxy, ethoxy, fluorine or chlorine, preferably hydrogen, methoxy, ethoxy or fluorine;
R⁴ hydrogen, fluorine or chlorine, preferably hydrogen or fluorine;
optionally in the form of the pharmaceutically acceptable acid addition salts, hydrates or solvates thereof, optionally in admixture with one or more physiologically acceptable excipients.

4) An inhalable powder according to claim 1 which contains one or more enantiomerically pure compounds of general formula 1, wherein
R¹ denotes hydrogen or fluorine;
R² denotes hydrogen;
R³ denotes methoxy, ethoxy or fluorine;
R⁴ denotes hydrogen;
optionally in the form of the pharmaceutically acceptable acid addition salts, hydrates or solvates thereof, optionally in admixture with one or more physiologically acceptable excipients.

5) An inhalable powder according to claim 1, which contains one or more enantiomerically pure compounds of formula 1, wherein
R¹ denotes hydrogen;
R² denotes hydrogen, fluorine or chlorine, preferably hydrogen or fluorine;
R³ denotes hydrogen;
R⁴ denotes hydrogen, fluorine or chlorine, preferably hydrogen or fluorine;
optionally in the form of the pharmaceutically acceptable acid addition salts, hydrates or solvates thereof, optionally in admixture with one or more physiologically acceptable excipients.

6) An inhalable powder according to claim 1 which contains one or more enantiomerically pure compounds of formula 1 in the form of their free bases.

7) An inhalable powder according to claim 1 which contain one or more enantiomerically pure compounds of formula 1 in the form of their pharmaceutically acceptable acid addition salts.

8) An inhalable powder according to claim 1, which contains one or more enantiomerically pure compounds of formula 1 in the form of their acid addition salts of general formula 1-HX, 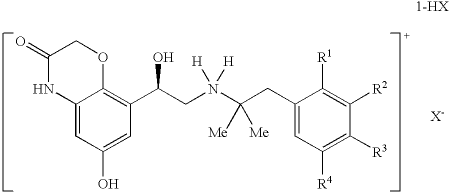
wherein
X⁻ denotes an anion with a single negative charge, preferably an anion with a single negative charge selected from the group consisting of chloride, bromide, iodide, sulphate, phosphate, methanesulphonate, nitrate, maleate, acetate, benzoate, citrate, salicylate, trifluoroacetate, fumarate, tartrate, oxalate, succinate, benzoate and p-toluenesulphonate,
and the groups R₁, R², R³ and R⁴ may have the meanings given in claims 1 to 5, optionally in the form of the tautomers, mixtures of the tautomers, hydrates or solvates thereof, as well as optionally in admixture with one or more physiologically acceptable excipients.

9) An inhalable powder according to claim 1, characterised in that the compounds of general formula 1 have the parameters of a characteristic particle size X₅₀ of from 0.1 μm to 10 μm and Q_{(5.8)} of more than 60%.

10) An inhalable powder according to claim 1, characterised in that they contain only one or more compounds of formula 1.

11) An inhalable powder according to claim 1, characterised in that they contain in addition to one or more compounds of general formula 1 at least one physiologically acceptable excipient.

12) An inhalable powder according to claim 11, characterised in that the physiologically acceptable excipient is selected from the group consisting of monosaccharides, disaccharides, oligo- and polysaccharides, polylactide/glycolide, polyalcohols, amino acids, chitosan, alkali metal and alkaline earth metal salts of stearic acid, salts and mixtures of these excipients with one another.

13) An inhalable powder according to claim 12, characterised in that the physiologically acceptable excipient is selected from the group comprising glucose, fructose, arabinose, lactose, saccharose, maltose, trehalose, maltodextrin, starch, cellulose, resomer, sorbitol, mannitol, xylitol, arginine hydrochloride, Mg-stearate, sodium chloride, calcium carbonate and mixtures of these excipients with one another.

14) An inhalable powder according to claim 1, characterised in that the ratio of compound of formula 1 to physiologically acceptable excipient is within the range from 5:100 to 1:100000.

15) An inhalable powder according to claim 1, characterised in that the physiologically acceptable excipient has an average particle size of about 17-120 μm.

16) A pharmaceutical composition comprising a compound of formula 1 according to claim 1 and a pharmaceutically acceptable carrier or excipient thereof.

17) A method of treating respiratory complaints comprising administering to a patient in need thereof a therapeutically effective amount of a compound of formula 1 according to claim 1.

18) A pharmaceutical formulation, characterised in that it contains an inhalable powder according to claim 1.
